(19) United States
(12) Patent Application Publication
(10) Pub. No.: US 2025/0034512 A1
(43) Pub. Date: Jan. 30, 2025

(54) PSEUDOALTEROMONAS AGARIVORANS STRAIN AND PREPARATION METHOD OF EXOPOLYSACCHARIDE (EPS) THEREFROM

(71) Applicant: QILU UNIVERSITY OF TECHNOLOGY (SHANDONG ACADEMY OF SCIENCES), Jinan, Shandong (CN)

(72) Inventors: Lujiang HAO, Jinan, Shandong (CN); Yuhao JU, Jinan, Shandong (CN); Wei WANG, Jinan, Shandong (CN); Kai SHAN, Jinan, Shandong (CN); Xiaofei ZHANG, Jinan, Shandong (CN); Ruiwen CAO, Jinan, Shandong (CN); Nan LIU, Jinan, Shandong (CN); Gangrui ZHANG, Jinan, Shandong (CN); Xingbao WANG, Jinan, Shandong (CN)

(21) Appl. No.: 18/275,380

(22) PCT Filed: Mar. 30, 2023
(86) PCT No.: PCT/CN2023/085038

§ 371 Date: Aug. 1, 2023

(51) Int. Cl. C12N 1/20; B01D 15/36; C12P 19/04; C12R 1/01

(52) U.S. Cl. CPC C12N 1/20; B01D 15/361; C12P 19/04; C12R 2001/01

(57) ABSTRACT

The present disclosure provides a Pseudoalteromonas agarivorans strain and a preparation method of an exopolysaccharide (EPS), and relates to the technical field of functional bacteria. In the present disclosure, the Pseudoalteromonas agarivorans strain HJWL2022 has a deposit number of CGMCC No. 26272. Fermentation, centrifugation, alcohol precipitation, protein removal, purification, and drying are conducted with the strain HJWL2022 to obtain a pure EPS. A proportion of a monosaccharide component in the product EPS is controlled by precisely controlling a pH value in a fermentation environment of the strain HJWL2022. Targeted control of a monosaccharide ratio is achieved to enhance a certain biological activity by adjusting fermentation conditions to change a composition of a bacterial EPS.

This application is a national stage application of International Patent Application No. PCT/CN2023/085038, filed on Mar. 30, 2023.

### TECHNICAL FIELD

The present disclosure belongs to the technical field of functional bacteria, and in particular relates to a Pseudoalteromonas agarivorans strain and a preparation method of an exopolysaccharide (EPS) therefrom.

### BACKGROUND

A variety of extracellular active substances produced by bacteria are endowed with important basic research and application development values due to unique biological characteristics. Bacteria respond to environmental factors such as ocean acidification pollution and climate warming by regulating the metabolism of extracellular substances. Moreover, the bacteria secrete exopolysaccharides (EPSs) with complex structures and regulatory activities to play an antagonistic role during self-protection. This may be a key mechanism for bacteria to adapt to complex and diverse marine environments. Bacterial EPS is a major component of extracellular polymers. The EPS adheres to the peptidoglycan layer or outer membrane through a hydration network structure, to stabilize the formation of biofilms and respond to environmental stress and antagonism, including environmental factors of temperature, pH, heavy metals, and dissolved oxygen. In this way, metabolic disorders such as osmotic pressure imbalance, cell dehydration, and membrane structure degeneration caused by the above-mentioned disturbances are avoided, thereby exerting the barrier function of bacteria. A composition ratio of monosaccharides in the EPS is affected by the disturbance of environmental factors. In recent years, with the development of comprehensive technologies such as gene proteomics at home and abroad, a stress regulation mechanism for the allosteric and active effects of EPS secreted by multi-bacteria tends to be increasingly clear and has become a research hotspot.

At present, most studies on EPS-producing bacteria ignore the influence of environmental factors on the EPS. Most of the current researches focus on EPS produced under fixed environmental factors. However, when microorganisms respond to environmental changes, little is known about how the products change. Moreover, the research on changed products is also extremely simple.

### SUMMARY

In order to solve the above problems, an objective of the present disclosure is to provide a Pseudoalteromonas agarivorans strain and a preparation method of an exopolysaccharide (EPS) therefrom. In the present disclosure, Pseudoalteromonas agarivorans is used as a production strain, and a proportion of a monosaccharide component in the product EPS is controlled by precisely controlling a fermentation environment.

To achieve the above objective, the present disclosure provides the following technical solutions:

The present disclosure provides a Pseudoalteromonas agarivorans strain HJWL2022, where the Pseudoalteromonas agarivorans strain HJWL2022 has been biologically preserved, with a deposit number of CGMCC No. 26272.

The present disclosure further provides a method for preparing an EPS from the Pseudoalteromonas agarivorans strain HJWL2022, including the following steps: inoculating the Pseudoalteromonas agarivorans strain HJWL2022 into a fermentation medium, conducting fermentation culture for 36 h, and removing bacterial cells to obtain a supernatant: where the supernatant includes the EPS; and
the fermentation medium includes the following components by concentration: 30.0 g/L to 45.0 g/L of glucose, 1.0 g/L to 2.0 g/L of ammonium sulfate, and 35.0 g/L of seasalt.

Preferably, the Pseudoalteromonas agarivorans strain HJWL2022 is subjected to activation and seed solution culture before the inoculating is conducted; and

a process of the seed solution culture includes: inoculating an activated Pseudoalteromonas agarivorans strain HJWL2022 into a Zobell 2216E seed medium for a marine bacterium, and conducting culture at 25° C. and 180 r/min for 8 h to obtain a seed solution.

Preferably, the Zobell 2216E seed medium for a marine bacterium is an improved Zobell 2216E seed medium with a pH value of 7.6 to 7.8, including the following components by concentration: 5.0 g/L of peptone, 1.0 g/L to 2.0 g/L of a yeast extract, and 35.0 g/L of seasalt.

Preferably, a seed solution of the Pseudoalteromonas agarivorans strain HJWL2022 is inoculated in the fermentation medium at an inoculum size of 8% to 10%.

Preferably, the fermentation culture is conducted at 25° C. and an initial pH value of 7 to 9.

Preferably, the method further includes: concentrating the supernatant, mixing an obtained concentrated supernatant with 95% ethanol, allowing a resulting mixture to stand and conducting centrifugation to collect a precipitate, and drying the precipitate to obtain a crude EPS.

Preferably, the concentrating refers to concentrating the supernatant to ⅓ of an original volume:
the concentrated supernatant is mixed with the 95% ethanol at a volume ratio of 1:3; and
the mixing is conducted by stirring.

Preferably, the centrifugation is conducted at 4° C. and 8,000 r/min for 10 min.

Preferably, the method further includes purification after the crude EPS is obtained: where the purification includes: removing a protein in the crude EPS, sequentially conducting ion exchange column chromatography, molecular sieve chromatography, and dialysis, and then drying to obtain a pure EPS.

Preferably, the ion exchange column chromatography conducts gradient elution with a NaCl solution using a cellulose aqueous solution as a filler:
the molecular sieve chromatography conducts gradient elution with a NaCl solution using dextran gel as a filler; and
the dialysis is conducted at a molecular weight cut-off of 8,000 Da to 14,000 Da.

The present disclosure further provides an EPS prepared by the method.

Preferably, the EPS includes mannose, rhamnose, and glucose as main components.

Preferably, the EPS further includes glucuronic acid, galacturonic acid, and galactose.

Preferably, the glucose and the mannose each are present in a form of pyranose.

The present disclosure further provides a method for adjusting a composition of an EPS by changing a fermentation parameter, including: adjusting an initial pH value of fermentation.

Preferably, under an initial pH value of fermentation at 7 to 9, a proportion of glucose decreases with an increase of the initial pH value of fermentation, and a proportion of mannose increases with an increase of the initial pH value of fermentation:

under an initial pH value of fermentation at 7 to 9, there are characteristic absorption peaks of a polysaccharide, α-D-glucopyranose, and α-D-mannopyranose obtained by infrared spectrum analysis; and under an initial pH value of fermentation at 9, there are further characteristic absorption peaks of β-D-mannopyranose and D-furanose obtained by the infrared spectrum analysis.

The present disclosure further provides use of the method in targeted control of a monosaccharide ratio of the EPS.

The present disclosure further provides use of the EPS in scavenging a hydroxyl radical, a 2,2-diphenyl-1-picrylhydrazyl (DPPH) radical, and an 2,2′-azino-bis (3-ethylbenzothiazoline-6-sulfonic acid) (ABTS) radical.

The present disclosure has the following beneficial effects:
In the present disclosure, the Pseudoalteromonas agarivorans strain HJWL2022 is used as a production strain. A proportion of a monosaccharide component in the product EPS is controlled by precisely controlling a pH value in a fermentation environment of the strain HJWL2022. This strain is subjected to fermentation, centrifugation, alcohol precipitation, protein removal, purification, and drying to obtain a final product. The results show that ratios of glucose and mannose in the EPS change regularly with an change of the pH value. The ratio of the glucose decreases with an increase of an initial pH value of fermentation, from 90.28% at pH=7 to 54.99% at pH=9; while the ratio of the mannose increases with the initial pH value of fermentation, from 6.6% at pH=7 to 25.72% at pH=9. Infrared spectrum analysis shows that the EPS prepared from the Pseudoalteromonas agarivorans strain HJWL2022 at different temperatures and initial fermentation pH values all have characteristic absorption peaks of a polysaccharide, α-D-glucopyranose, and α-D-mannopyranose. However, the EPS produced under initial fermentation pH=9 also have characteristic absorption peaks of β-D-mannopyranose and D-furanose. This indicates that changes in the initial pH value of fermentation affect the sugar ring form and glycosidic bond configuration of EPS. An antioxidant activity of the strain is evaluated and compared by measuring a scavenging capacity of the EPS of Pseudoalteromonas agarivorans strain HJWL2022 on hydroxyl radicals. DPPH radicals, and ABTS radicals under different initial pH values of fermentation. Experimental results show that the EPS produced under initial fermentation pH=9 has a higher scavenging rate for the hydroxyl radicals. DPPH radicals, and ABTS radicals than that produced under initial fermentation pH=7 and pH=8 at the highest concentration. Targeted control of a monosaccharide ratio is achieved to enhance a certain biological activity by adjusting fermentation conditions to change a composition of a bacterial EPS.

### Deposit of Biological Material

In the present disclosure, the Pseudoalteromonas agarivorans strain HJWL2022 was deposited on December 26, 2022 in the China General Microbiological Culture Collection Center (CGMCC), Institute of Microbiology, Chinese Academy of Sciences, at NO. 1 West Beichen Road. Chaoyang District. Beijing 100101, with a deposit number of CGMCC No. 26272.

### BRIEF DESCRIPTION OF THE DRAWINGS

FIGS. 1A-D show liquid chromatograms of EPS under different pH conditions: in the figure. FIG. 1A represents a standard: FIG. 1B represents initial pH=7 of a fermentation broth:

FIG. 1C represents initial pH=8 of the fermentation broth: FIG. 1D represents initial pH=9 of the fermentation broth; and in the chromatograms, 1 to 7 are mannose, rhamnose, glucuronic acid, galacturonic acid, glucose, galactose, and arabinose in sequence:

FIGS. 2A-C show infrared spectrograms of EPS under different pH conditions; in the figure, a represents initial pH=7 of a fermentation broth: b represents initial pH=8 of the fermentation broth; and c represents initial pH=9 of the fermentation broth:

FIG. 3 shows molecular weight distribution of purified EPS by high-performance gel permeation chromatography (HP-GPC):

FIG. 4 shows a scavenging capacity of hydroxyl radicals by EPS under different initial pH values of fermentation:

FIG. 5 shows a scavenging capacity of DPPH radicals by EPS under different initial pH values of fermentation; and

FIG. 6 shows a scavenging capacity of ABTS radicals by EPS under different initial pH values of fermentation.

### DETAILED DESCRIPTION OF THE EMBODIMENTS

The present disclosure provides a Pseudoalteromonas agarivorans strain HJWL2022, where the Pseudoalteromonas agarivorans strain HJWL2022 has been biologically preserved, with a deposit number of CGMCC No. 26272.

In the present disclosure, the strain HJWL2022 is isolated and screened from a seedling plate of Haliotis discus hannai in Dajijia Breeding Plant in Rongcheng City, Shandong Province. Their colonies are milky white and round, have bulges and rounded edges, and are opaque and non-migratory:

In the present disclosure, the strain HJWL2022 is preferably stored on a paraffin slant medium.

The present disclosure further provides a method for preparing an EPS from the Pseudoalteromonas agarivorans strain HJWL2022, including the following steps: inoculating the Pseudoalteromonas agarivorans strain HJWL2022 into a fermentation medium, conducting fermentation culture for 36 h, and removing bacterial cells to obtain a supernatant: where the supernatant includes the EPS; and

the fermentation medium includes the following components by concentration: 30.0 g/L to 45.0 g/L of glucose, 1.0 g/L to 2.0 g/L of ammonium sulfate, and 35.0 g/L of seasalt.

In the present disclosure, the Pseudoalteromonas agarivorans strain HJWL2022 is preferably subjected to activation and seed solution culture before the inoculating is conducted; and a process of the seed solution culture includes: inoculating an activated Pseudoalteromonas agarivorans strain HJWL2022 into a Zobell 2216E seed medium for a marine bacterium, and conducting culture at 25° C. and 180 r/min for 8 h to obtain a seed solution. The activation includes preferably: allowing the Pseudoalteromonas agarivorans strain HJWL2022 stored on the paraffin slant medium to stand at 25° C. for 24 h. inoculating into the Zobell 2216E seed medium, and conducting culture in a shaker at 25° C. and 180 r/min for 12 h to complete the activation. A bacterial solution of the activated HJWL2022 strain is inoculated into the Zobell 2216E seed medium of a marine bacterium, at an inoculum size of preferably 8% to 10%. The Zobell 2216E seed medium for a marine bacterium is preferably an improved Zobell 2216E seed medium with a pH value of 7.6 to 7.8, including the following components by concentration: 5.0 g/L of peptone, 1.0 g/L to 2.0 g/L of a yeast extract, and 35.0 g/L of seasalt.

In the present disclosure, a seed solution of the Pseudoalteromonas agarivorans strain HJWL2022 is preferably inoculated into the fermentation medium at an inoculum size of 8% to 10% to allow fermentation culture. The fermentation culture is preferably conducted at 25° C. and an initial pH value of 7 to 9 for 36 h.

In the present disclosure, bacterial cells are removed after the fermentation culture is completed. Preferably, an obtained fermentation broth is centrifuged at 4° C., and 4.000 r/min for 5 min to remove the bacterial cells, thereby obtaining a supernatant containing EPS.

In the present disclosure, the method further includes obtaining a crude EPS from the supernatant through alcohol precipitation, and obtaining a pure EPS after protein removal and purification based on the crude EPS.

In the present disclosure, the alcohol precipitation includes preferably: concentrating the supernatant, mixing an obtained concentrated supernatant with 95% ethanol, allowing a resulting mixture to stand and conducting centrifugation to collect a precipitate, and drying the precipitate to obtain a crude EPS. The concentration is preferably conducted in a rotary evaporator. When concentrated to one third of an original volume, a resulting concentrated supernatant is mixed with 95% ethanol. The mixing is preferably conducted under the stirring of a magnetic stirrer, and specifically includes: slowly adding 3 times a volume of the 95% ethanol into the concentrated supernatant, and putting an obtained mixture in a refrigerator overnight to make the precipitation complete. The ethanol is preferably 95% ethanol. Preferably, a sample with complete precipitation is taken out, a supernatant is discarded, a remaining product is centrifuged at 4° C., and 8.000 r/min for 10 min, a resulting precipitate is collected, and then placed at room temperature to allow the alcohol to have evaporation and air-drying, thereby obtaining a crude polysaccharide.

In the present disclosure, a Sevage reagent is preferably used for protein removal. The Sevage reagent preferably includes a mixed solution of chloroform and n-butanol, and the chloroform and the n-butanol are at a volume ratio of 4:1. The protein removal is preferably conducted in a separatory funnel. Deionized water and the crude EPS are configured to form an aqueous solution of the crude EPS with an appropriate concentration, and poured into the separatory funnel. 3 times a volume of the Sevage reagent is added and shaken fully. A resulting mixture is allowed to stand for 10 min, a valve of the separatory funnel is opened, and the Sevage reagent and denatured protein precipitates are removed by filtration. The above operations are repeated 4 to 5 times until no denatured protein appears. After the protein removal, residual Sevage reagent is preferably also removed.

In the present disclosure, the purification includes preferably ion exchange column chromatography, molecular sieve chromatography; and dialysis in sequence. The ion exchange column chromatography conducts gradient elution preferably with a NaCl solution using a cellulose aqueous solution as a filler: the molecular sieve chromatography conducts gradient elution preferably with a NaCl solution using dextran gel as a filler; and
the dialysis is preferably conducted at a molecular weight cut-off of 8,000 Da to 14,000 Da.

The present disclosure further provides an EPS prepared by the method.

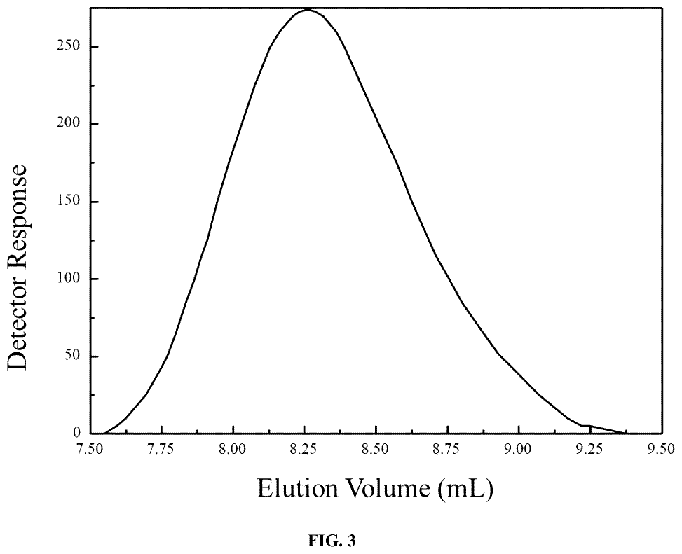

In the present disclosure, the EPS includes preferably mannose, rhamnose, and glucose as main components, and further includes a small amount of glucuronic acid, galacturonic acid, and galactose. The glucose and the mannose mainly exist in a form of pyranose, and their molecular weights are shown in FIG. 3.

The present disclosure further provides a method for adjusting a composition of an EPS by changing a fermentation parameter, including: adjusting an initial pH value of fermentation.

In the present disclosure, targeted control of a proportion of a monosaccharide component in the product EPS is controlled by precisely controlling a pH value in a fermentation environment of the strain HJWL2022. For example, under an initial pH value of fermentation at 7 to 9, a proportion of glucose decreases with an increase of the initial pH value of fermentation, and a proportion of mannose increases with an increase of the initial pH value of fermentation: under an initial pH value of fermentation at 7 to 9, there are characteristic absorption peaks of a polysaccharide, α-D-glucopyranose, and α-D-mannopyranose obtained by infrared spectrum analysis; and under an initial pH value of fermentation at 9, there are further characteristic absorption peaks of β-D-mannopyranose and D-furanose obtained by the infrared spectrum analysis.

The present disclosure further provides use of the method in targeted control of a monosaccharide ratio of the EPS.

The present disclosure further provides use of the EPS in scavenging a hydroxyl radical, a 2,2-diphenyl-1-picrylhydrazyl (DPPH) radical, and an 2,2′-azino-bis (3-ethylbenzothiazoline-6-sulfonic acid) (ABTS) radical.

In order to further illustrate the present disclosure, the Pseudoalteromonas agarivorans strain and the preparation method of an EPS provided by the present disclosure are described in detail below in connection with accompanying drawings and examples, but these examples should not be understood as limiting the claimed scope of the present disclosure.

Unless otherwise specified, materials, reagents, instruments, and methods used in examples of the present disclosure can all be obtained according to the conventional content in this field:
1. A seed medium is a slightly-modified Zobell 2216E medium for a marine bacterium at a pH value adjusted to 7.7, including: 5.0 g of peptone, 1.2 g of a yeast extract, 35.0 g of seasalt, and 1 L of deionized water.
2. A fermentation medium has a pH value of 8, including: 45.5 g of glucose, 2.0 g of ammonium sulfate, 35.0 g of seasalt, and 1 L of deionized water.

TABLE 1
Main reagents and raw materials

| | Specifi- | |
|---|---|---|
| Name | cation | Factory |
| Peptone | AR | Nantong Huge Bio-Tech Co., Ltd. |
| Yeast extract | AR | Shanghai Yuan Peptide Biotechnology |
| | | Co., Ltd. |
| Seasalt | AR | Jiangxi Yantong Technology Co., Ltd. |
| Glucose | AR | Tianjin Damao Chemical Reagent |
| | | Factory |
| Ammonium sulfate | AR | Sinopharm Chemical Reagent Co., Ltd. |
| Phenol | AR | Hongyan Reagent Factory, Hedong |
| | | District, Tianjin |
| Trichloromethane | AR | Nanjing Kangke Chemical Co., Ltd. |
| 95% ethanol | AR | Nanjing Chemical Reagent Co., Ltd. |
| n-butanol | AR | Dongguan Huaxin Chemical |
| | | Technology Co., Ltd. |
| Sodium chloride | AR | Tianjin Kaitong Chemical Reagent Co., |
| | | Ltd. |
| DEAE-52 cellulose | BC | Shanghai Mengya Biotechnology Co., |
| | | Ltd. |
| Dextran gel G-100 | BC | Beijing Ruida Henghui Technology |
| | | Development Co., Ltd. |
| Sodium hydroxide | AR | Sinopharm Chemical Reagent Co., Ltd. |
| Potassium bromide | SP | Tianjin Bojun Technology Co., Ltd. |
| 1-phenyl-3-methyl- | AR | Shanghai Macklin Biochemical Co., Ltd. |
| 5-pyrazolone (PMP) | | |
| Ammonium acetate | AR | Wuxi Olite Chemical Co., Ltd. |
| Acetonitrile | HPLC | Shanghai Acmec Biochemical Co., Ltd. |
| Methanol | HPLC | Shanghai Acmec Biochemical Co., Ltd. |
| Silver nitrate | AR | Shanghai Macklin Biochemical Co., Ltd. |
| DPPH | AR | Shanghai Aladdin Biochemical |
| | | Technology Co., Ltd. |
| ABTS | AR | Shanghai Aladdin Biochemical |
| | | Technology Co., Ltd. |
| Potassium persulfate | AR | Sinopharm Chemical Reagent Co., Ltd. |
| Ferrous sulfate | AR | Sinopharm Chemical Reagent Co., Ltd. |
| Salicylic acid | AR | Tianjin Dingshengxin Chemical Co., Ltd. |
| Anhydrous ethanol | AR | Tianjin Fuyu Chemical Co., Ltd. |
| Hydrogen peroxide | AR | Shanghai Aladdin Biochemical |
| solution | | Technology Co., Ltd. |

TABLE 2
Main instruments and equipment

| Name | Factory |
|---|---|
| Chromatographic column | Wuhan Jingcheng Biotechnology |
| (φ = 2.6 × 40 cm and | Co., Ltd. |
| φ = 1.6 × 100 cm) | |
| Constant flow pump | Shanghai Jingke Industrial Co., Ltd. |
| Automatic fraction collector | Cany Precision Instrument Co., Ltd. |
| Magnetic stirrer | Shenzhen Boda Jingke Biotechnology Co., |
| | Ltd. |
| pH meter | Shanghai Sanxin & Apera Instruments |
| Rotary evaporator | Shanghai Yuezhong Instrument & |
| | Equipment Co., Ltd. |
| High-speed refrigerated | Eppendorf China |
| centrifuge Centrifuge5804R | |
| Constant-temperature | Jiangsu Chunhui Environmental Protection |
| water bath | Equipment Co., Ltd. |
| Constant-temperature | Shanghai Zhicheng Analytical |
| shaking incubator | Instrument Manufacturing Co., Ltd. |
| Refrigerator | Hisense Group Co., Ltd. |
| Vacuum freeze-dryer | Jiangsu Tianling Instrument Co., Ltd. |
| UV-Vis spectrophotometer | Shanghai Yuanxi Instrument Co., Ltd. |
| LC-20A high-performance | Shimadzu Corporation |
| liquid chromatograph | |
| Infrared spectrometer | PerkinElmer Inc. |
| Spectrum Two | |
| Analytical balance | Tianjin Jingtuo Instrument |
| | Technology Co., Ltd. |
| Microplate reader | Shanghai Yuanxi Instrument Co., Ltd. |

### Example 1

(1) A Pseudoalteromonas agarivorans strain HJWL2022 was activated, inoculated into a Zobell 2216E seed medium of a marine bacterium, and cultured at 25° C., and 180 r/min for 8 h.
(2) An activated strain obtained in step (1) was transferred to fermentation media with initial pH values of 7, 8, and 9 at an inoculum size of 8%.
(3) The strain cultured in step (2) was subjected to shaking culture at 180 r/min for 36 h in a shaker at a constant temperature of 25° C., and then centrifuged at 4° C., and 4,000 r/min for 5 min to remove bacterial cells. A resulting supernatant was poured into a rotary evaporator, concentrated to one third of its original volume, and poured into a beaker. Under stirring with a magnetic stirrer, 95% ethanol 3 times a volume of an obtained concentrated solution was slowly added to the concentrated solution, and put in a refrigerator overnight to make precipitation complete.
(4) The sample overnight in step (3) was taken out, a supernatant was discarded, a remaining product was centrifuged at 4° C., and 8,000 r/min for 10 min, a resulting precipitate was collected, and then placed at room temperature to allow the alcohol to have evaporation and air-drying, and weighed to obtain a crude polysaccharide product.
(5) The chloroform and n-butanol were mixed at a ratio of 4:1 to obtain a Sevage reagent. The crude exopolysaccharide product obtained in step (4) was dissolved with deionized water to obtain an aqueous solution of the crude EPS with an appropriate concentration, and poured into the separatory funnel. 3 times a volume of the Sevage reagent was added and shaken fully. A resulting mixture was allowed to stand for 10 min, a valve of the separatory funnel was opened, and the Sevage reagent and denatured protein precipitates were removed by filtration. The above operations were repeated 4 to 5 times until no denatured protein appeared. The residual Sevage reagent in the crude EPS aqueous solution was removed by a rotary evaporator, for later use.
(6) 60 g of DEAE-52 cellulose was added to 500 mL of deionized water, stirred evenly with a glass rod, allowed to stand at room temperature for 24 h, and a turbid liquid floating on an upper layer was discarded. The processes were repeated 2 to 3 times. A treated DEAE-52 cellulose filler was degassed and slowly added to a chromatographic column (φ=2.6×40) cm) to a distance of 5 cm from a column mouth, and natural settlement was conducted to ensure that a surface of the filler was smooth and there were no fine air bubbles in the chromatographic column. The chromatographic column was equilibrated with 2 column volumes of deionized water. The EPS aqueous solution treated in step (5) was added slowly along an inner wall of the chromatographic column, and gradient elution was conducted with a (0-2) mol/L NaCl solution at a flow rate of 2 mL/min. Detection was conducted by a phenol-sulfuric acid method, and eluted fractions were combined. After the elution was completed, the DEAE-52 cellulose was regenerated by an alkali-acid-alkali method. The cellulose was soaked in a 0.5 mol/L NaOH solution for 30 min, suction-filtered, and rinsed with deionized water until neutral, then soaked in a 0.5 mol/L HCl solution for 30 min, suction-filtered, and rinsed with deionized water until neutral. The DEAE-52 cellulose was suction-filtered and washed with 0.5 mol/L NaOH in the same way as above until the residual impurities were removed.
(7) 50 g of a dextran gel dry powder (Sephadex G-100) was immersed in 60% ethanol for 24 h, stirred constantly to ensure that the gel was fully swollen, and the residual ethanol was washed away with deionized water. A treated dextran gel dry powder (Sephadex G-100) was degassed and slowly added to a chromatographic column (φ=1.6×100 cm) to a distance of 5 cm from a column mouth, and natural settlement was conducted. The chromatographic column was equilibrated with 2 column volumes of deionized water. The EPS aqueous solution treated by ion exchange column chromatography in step (6) was added slowly along an inner wall of the chromatographic column, and gradient elution was conducted with a (0-0.2) mol/L NaCl solution at a flow rate of 0.5 mL/min. Detection was conducted by a phenol-sulfuric acid method, and eluted fractions were combined.
(8) The EPS aqueous solution treated in step (7) was dialyzed with a dialysis bag with a molecular weight cut-off of 8.000 Da to 14,000 Da. The dialysis bag was cut into small sections of about 20 cm with sterile scissors, boiled in deionized water for 20 min, cooled, and then rinsed with deionized water. One end of the dialysis bag was clamped with a dialysis clamp, and a small amount of deionized water was added to check for fluid leakage. The EPS aqueous solution was added to a height that did not exceed ⅔ of the dialysis bag. The other end of the dialysis bag was clamped with the dialysis clamp to check for fluid leakage again. The deionized water used in dialysis was replaced once every 6 h, and whether the dialysis was complete was determined by adding an AgNO₃ solution dropwise to the deionized water. If there was no white precipitate, the dialysis could be terminated, and pure EPS of Pseudoalteromonas agarivorans strain HJWL2022 could be obtained by freeze-drying in a vacuum freeze-dryer.
(9) Preparation of a standard solution of mixed monosaccharides: 0.01802 g of glucose, 0.01802 g of galactose, 0.01802 g of mannose, 0.01822 g of rhamnose, 0.01941 g of glucuronic acid, 0.02122 g of galacturonic acid, and 0.01501 g of arabinose were mixed and diluted to 50 ml with ultrapure water, to obtain a 2 mmol/L mixed monosaccharide standard solution.

Hydrolysis of EPS: 25 mg of an EPS sample was poured into 10 mL of 1 mol/L H₂SO₄, sealed and heated in a water bath at 100° C., for 8 h, then taken out. After centrifugation at 6,000 r/min for 5 min, a small amount of 2 mol/L NaOH was added to an obtained supernatant to make a final pH value to be 7, and 5 mL of an obtained hydrolyzate was mixed with 5 mL of deionized water for derivatization.

Derivatization of PMP: 50 μL of an EPS hydrolysis sample solution and the standard solution of mixed monosaccharides were mixed with a reaction system including 50 μL of 0.5 mol/L PMP-methanol solution, 0.3 mol/L of NaOH, and 100 μL of ultrapure water. A resulting mixture was extracted with 900 μL of chloroform, vortexed for 10 s, centrifuged at 4,000 r/min for 5 min, and a turbid lower layer was discarded. The extraction was repeated 3 times, and an obtained aqueous phase was filtered through a 0.22 μm filter membrane for later use.

Relevant parameter setting of HPLC: a Shimadzu InertSustain C18 chromatographic column was selected, where a mobile phase was ammonium acetate buffer solution-acetonitrile with a volume ratio of 80:20 and pH=5.5, a flow rate was set to 1 mL/min, a column temperature was 30° C., and detection was conducted using a UV detector with a detection wavelength of 245 nm.

The EPS of Pseudoalteromonas agarivorans strain HJWL2022 was extracted and purified by water extraction and alcohol precipitation, protein removal by Sevage method, ion exchange chromatography, and dextran gel chromatography under different initial pH values of fermentation. The monosaccharide composition, sugar ring form, and glycosidic bond configuration of EPS were determined by HPLC and Fourier transform infrared spectroscopy, so as to analyze the influence of environmental factors on the yield and structure of EPS.

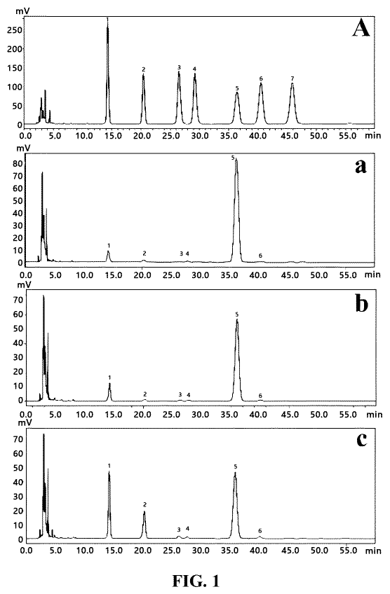

The experimental results showed that the initial pH value of fermentation broth could affect a crude EPS yield of Pseudoalteromonas agarivorans strain HJWL2022. The monosaccharide compositions of EPS prepared from the Pseudoalteromonas agarivorans strain HJWL2022 at different initial pH values were mannose, rhamnose, glucuronic acid, galacturonic acid, glucose, and galactose. The main components were glucose, mannose, and rhamnose. The ratios of mannose and rhamnose showed a decreasing trend with the increase of the initial pH value of fermentation, while the ratio of glucose with the initial pH value of fermentation showed an opposite trend (Table 3 and FIGS. 1A-D).

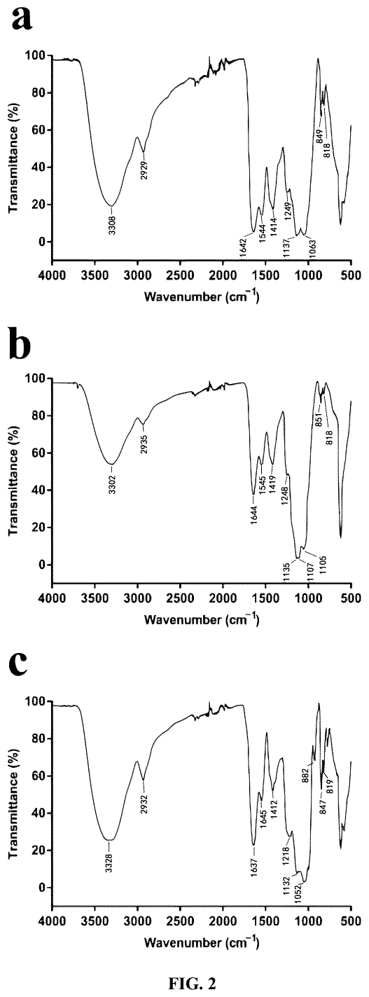

Infrared spectrum analysis showed that the EPS prepared from the Pseudoalteromonas agarivorans strain HJWL2022 at different initial fermentation pH values all had characteristic absorption peaks of a polysaccharide, α-D-glucopyranose, and α-D-mannopyranose. However, the EPS produced under initial fermentation pH=9 also had characteristic absorption peaks of β-D-mannopyranose and D-furanose. This indicated that changes in the initial pH value of fermentation affected the sugar ring form and glycosidic bond configuration of EPS (FIGS. 2A-C).

TABLE 3
Effect of pH change on monosaccharide composition and ratio of EPS

Initial pH of

| fermentation | Monosaccharide composition and area integral percentage/% | | | | | |
|---|---|---|---|---|---|---|
| broth | Man | Rha | GlcUA | GlaUA | Glc | Gla |
| Ph = 7 | 6.66 ± 0.96 | 1.81 ± 0.29 | 0.15 ± 0.06 | 0.56 ± 0.13 | 90.28 ± 0.82 | 0.54 ± 0.26 |
| pH = 8 | 9.16 ± 0.49 | 1.38 ± 0.26 | 0.59 ± 0.04 | 0.73 ± 0.02 | 86.87 ± 0.42 | 1.03 ± 0.09 |
| pH = 9 | 25.72 ± 1.13 | 13.9 ± 0.47 | 1.58 ± 0.56 | 0.5 ± 0.22 | 54.99 ± 2.29 | 3.31 ± 0.84 |

### Example 2

(1) A scavenging capacity of EPS produced by Pseudoalteromonas agarivorans strain HJWL2022 on hydroxyl radicals was determined at different initial pH values of fermentation. 0.1 mL each of 9 mmol/L FeSO₄ solution, 9 mmol/L salicylic acid-ethanol solution, and sample solutions to be tested of EPS with different concentrations (0.2, 0.4, 0.6, 0.8, and 1 mg/mL) were added into a 96-well plate, mixed thoroughly, and added with 0.1 mL of 1 mmol/L H₂O₂ solution to allow a reaction by incubating in a constant-temperature water bath at 37° C., for 30 min. An absorbance measured at 510 nm by a microplate reader was recorded as As; an absorbance measured by replacing salicylic acid-ethanol solution with 0.1 mL deionized water was recorded as Ae as a background group; and an absorbance measured by replacing the sample solution to be tested with 0.1 mL deionized water was recorded as Ac as a blank group. Each group of experiments was repeated 3 times to obtain an average, and a scavenging rate was calculated according to the following formula:

$$Hydroxyl radical scavenging rate ( \% ) = ( 1 - ( As - Ae ) / Ac ) \times 100$$

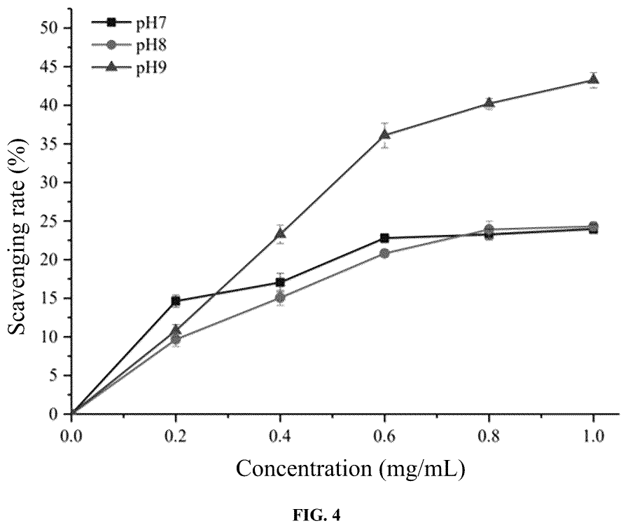

The experimental results were shown in FIG. 4. When the initial condition was pH=9, the concentration of EPS produced was at (0-0.6) mg/mL. With the increase of concentration, the scavenging rate of EPS to hydroxyl radicals increased rapidly, and the scavenging rate was higher than that of EPS produced under the initial conditions of pH=7 and pH=8. When the concentration was higher than 0.6 mg/mL, the scavenging rate of EPS produced at the initial condition of pH=9 increased slowly for hydroxyl radicals, but was still higher than that produced at the initial condition of pH=7 and pH=8.
(2) A scavenging capacity of EPS produced by Pseudoalteromonas agarivorans strain HJWL2022 on DPPH radicals was at different initial pH values of fermentation. 50 μL of sample solutions to be tested of EPS with different concentrations (0.2, 0.4, 0.6, 0.8, and 1 mg/mL), 25 μL of 0.4 mmol/L DPPH-ethanol solution, and 100 μL of deionized water were sequentially added to a 96-well plate, mixed thoroughly, and reacted at a constant temperature of 30° C., in the dark for 30 min. An absorbance measured at 517 nm by a microplate reader was recorded as As: an absorbance measured by replacing DPPH-ethanol solution with 0.1 mL anhydrous ethanol was recorded as Ae as a background group; and an absorbance measured by replacing the sample solution to be tested with 0.1 mL deionized water was recorded as Ac as a blank group. Each group of experiments was repeated 3 times to obtain an average, and a scavenging rate was calculated according to the following formula:

$$DPPH radical scavenging rate ( \% ) = ( 1 - ( As - Ae ) / Ac ) \times 100$$

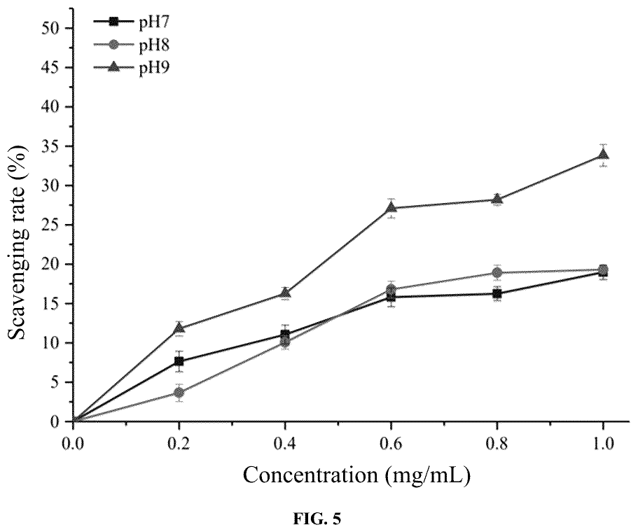

The experimental results were shown in FIG. 5. The concentration of EPS produced by Pseudoalteromonas agarivorans strain HJWL2022 at different initial pH values of fermentation was positively correlated with the scavenging rate of DPPH radicals, and the scavenging rate increased with the increase of EPS concentration. In the concentration range of the experiment, the scavenging rate of EPS produced under the initial condition of pH=9 to DPPH radicals was higher than that produced under the initial conditions of pH=7 and pH=8.
(3) A scavenging capacity of EPS produced by Pseudoalteromonas agarivorans strain HJWL2022 on ABTS radicals was at different initial pH values of fermentation. 88 μL of a K₂S₂O₈ stock solution (2.6 mmol/L) was mixed with 5 mL of an ABTS solution (7 mmol/L), and allowed to stand in the dark at room temperature for 12 h to 14 h to obtain an ABTS working solution. The working solution was diluted with an appropriate amount of deionized water until an absorbance at 734 nm was about 0.70±0.02 for later use. 20 μL of sample solutions to be tested of EPS with different concentrations (0.2, 0.4, 0.6, 0.8, and 1 mg/mL) were added to a 96-well plate, then 200 μL of the diluted ABTS solution was added to mix well, and allowed to stand at room temperature for 6 min. An absorbance measured at 734 nm by a microplate reader was recorded as As; an absorbance measured by 200 μL of deionized water instead of the ABTS dilution was recorded as Ae as a background group; and an absorbance measured by 20 μL of deionized water instead of the sample solution to be tested was recorded as Ac as a blank group. Each group of experiments was repeated 3 times to obtain an average, and a scavenging rate was calculated according to the following formula:

$$ABTS radical scavenging rate ( \% ) = ( 1 - ( As - Ae ) / Ac ) \times 100$$

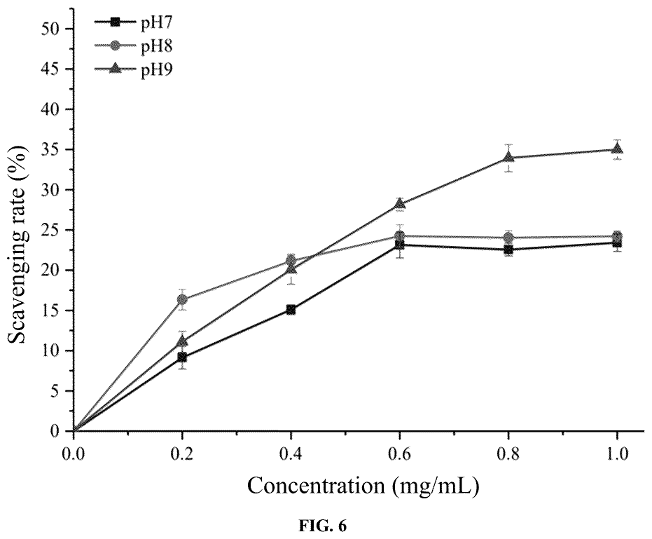

The experimental results were shown in FIG. 6. The concentration of EPS produced by Pseudoalteromonas agarivorans strain HJWL2022 at different initial pH values of fermentation was positively correlated with the scavenging rate of ABTS radicals, and the scavenging rate increased with the increase of EPS concentration. When the EPS concentration was (0-0.4) mg/ml and the initial condition was pH=8, the scavenging rate of EPS for ABTS radicals was higher than that of EPS produced under the initial conditions of pH=7 and pH=9. When the concentration was higher than 0.4 mg/mL, the scavenging rate of EPS produced at the initial condition of pH=9 increased rapidly for ABTS radicals, and was higher than that produced at the initial condition of pH=7 and pH=8. In the concentration range of the experiment, the scavenging rate of EPS produced under the initial condition of pH=8 to ABTS radicals was always higher than that produced under the initial condition of pH=7.

Although the present disclosure has been described in detail through the above examples, the examples are only a part rather than all of the examples of the present disclosure. All other examples obtained by persons based on these examples without creative efforts shall fall within a protection scope of the present disclosure.

1. A Pseudoalteromonas agarivorans strain HJWL2022, wherein the Pseudoalteromonas agarivorans strain HJWL2022 has a deposit number of CGMCC No. 26272.

2. A method for preparing an exopolysaccharide (EPS) from the Pseudoalteromonas agarivorans strain HJWL2022 according to claim 1, comprising the following steps: inoculating the Pseudoalteromonas agarivorans strain HJWL2022 into a fermentation medium, conducting fermentation culture for 36 h, and removing bacterial cells to obtain a supernatant; wherein the supernatant comprises the EPS; and
the fermentation medium comprises the following components by concentration: 30.0 g/L to 45.0 g/L of glucose, 1.0 g/L to 2.0 g/L of ammonium sulfate, and 35.0 g/L of seasalt.

3. The method according to claim 2, wherein the Pseudoalteromonas agarivorans strain HJWL2022 is subjected to activation and seed solution culture before the inoculating is conducted; and
a process of the seed solution culture comprises: inoculating an activated Pseudoalteromonas agarivorans strain HJWL2022 into a Zobell 2216E seed medium, and conducting culture at 25° C., and 180 r/min for 8 h to obtain a seed solution.

4. The method according to claim 3, wherein the Zobell 2216E seed medium for a marine bacterium is an improved Zobell 2216E seed medium with a pH value of 7.6 to 7.8, comprising the following components by concentration: 5.0 g/L of peptone, 1.0 g/L to 2.0 g/L of a yeast extract, and 35.0 g/L of seasalt.

5. The method according to claim 2, wherein a seed solution of the Pseudoalteromonas agarivorans strain HJWL2022 is inoculated in the fermentation medium at an inoculum size of 8% to 10%.

6. The method according to claim 2, wherein the fermentation culture is conducted at 25° C., and an initial pH value of 7 to 9.

7. The method according to claim 2, further comprising: concentrating the supernatant, mixing an obtained concentrated supernatant with 95% ethanol, allowing a resulting mixture to stand and conducting centrifugation to collect a precipitate, and drying the precipitate to obtain a crude EPS.

8. The method according to claim 7, wherein the concentrating refers to concentrating the supernatant to ⅓ of an original volume;
the concentrated supernatant is mixed with the 95% ethanol at a volume ratio of 1:3; and
the mixing is conducted by stirring.

9. The method according to claim 7, wherein the centrifugation is conducted at 4° C., and 8,000 r/min for 10 min.

10. The method according to claim 7, further comprising purification after the crude EPS is obtained; wherein the purification comprises: removing a protein in the crude EPS, sequentially conducting ion exchange column chromatography, molecular sieve chromatography, and dialysis, and then drying to obtain a pure EPS.

11. The method according to claim 10, wherein the ion exchange column chromatography conducts gradient elution with a NaCl solution using a cellulose aqueous solution as a filler;
the molecular sieve chromatography conducts gradient elution with a NaCl solution using dextran gel as a filler; and
the dialysis is conducted at a molecular weight cut-off of 8,000 Da to 14,000 Da.

12. An EPS prepared by the method according to claim 2.

13. The EPS according to claim 12, comprising mannose, rhamnose, and glucose as main components.

14. The EPS according to claim 12, further comprising glucuronic acid, galacturonic acid, and galactose.

15. The EPS according to claim 13, wherein the glucose and the mannose each are present in a form of pyranose.

16. A method for adjusting a composition of an EPS by changing a fermentation parameter, comprising: adjusting an initial pH value of fermentation.

17. The method according to claim 16, wherein under an initial pH value of fermentation at 7 to 9, a proportion of glucose decreases with an increase of the initial pH value of fermentation, and a proportion of mannose increases with an increase of the initial pH value of fermentation;
under an initial pH value of fermentation at 7 to 9, there are characteristic absorption peaks of a polysaccharide, α-D-glucopyranose, and α-D-mannopyranose obtained by infrared spectrum analysis; and under an initial pH value of fermentation at 9, there are further characteristic absorption peaks of β-D-mannopyranose and D-furanose obtained by the infrared spectrum analysis.

18. A process for directional control of a monosaccharide ratio of an EPS, wherein the process is the method for adjusting a composition of an EPS by changing a fermentation parameter according to claim 16.

19. A method for scavenging a hydroxyl radical, a 2,2-diphenyl-1-picrylhydrazyl (DPPH) radical, and an 2,2′-azino-bis (3-ethylbenzothiazoline-6-sulfonic acid) (ABTS) radical using the EPS according to claim 12.

20. The EPS according to claim 13, further comprising glucuronic acid, galacturonic acid, and galactose.
